(19) United States
(12) Patent Application Publication
(10) Pub. No.: US 2026/0364065 A1
(43) Pub. Date: Nov. 19, 2026

(54) Repurposing Usage of Posaconazole Delayed Release Tablet for Alzheimer Disease

(71) Applicants: Xuanzhong Ni, Campbell, CA (US); Caoning Zeng, Campbell, CA (US)

(72) Inventors: Xuanzhong Ni, Campbell, CA (US); Caoning Zeng, Campbell, CA (US)

(21) Appl. No.: 19/211,222

(22) Filed: May 18, 2025

(51) Int. Cl. A61K 31/496; A61K 9/20; A61P 25/28

(52) U.S. Cl. CPC A61K 31/496; A61K 9/2009; A61K 9/2018; A61K 9/2054; A61P 25/28

(57) ABSTRACT

The present invention is a Repurposing Usage of Posaconazole Delayed-release Tablet for Alzheimer's disease. This new use capitalizes on Posaconazole's neuroprotective, anti-inflammatory, and antioxidant properties, offering potential benefits for patients suffering from Alzheimer's disease.

Reference: This application claims the benefit of U.S. Provisional Application No. 63/649437, filed on date May 19, 2024, the entire contents of which are incorporated herein by reference.

### BACKGROUND OF THE INVENTION

Alzheimer's disease (AD) is a progressive neurodegenerative disorder that primarily affects the elderly and is characterized by cognitive decline, memory loss, confusion, and impaired ability to perform daily activities. Alzheimer's disease is linked to the accumulation of amyloid-beta plaques and tau tangles in the brain, leading to neuronal dysfunction and death. Currently, treatments for Alzheimer's disease are limited, focusing on symptom management rather than halting or reversing the disease's progression.

Posaconazole is a well-known antifungal agent with broad-spectrum activity, originally developed to treat invasive fungal infections. It is known to exhibit anti-inflammatory, neuroprotective, and immunomodulatory properties. Recent research has suggested that Posaconazole may have potential beyond its antifungal activity, including effects on brain function, neuroinflammation, and neuronal health. However, there is a lack of research exploring the potential of Posaconazole specifically for the treatment of Alzheimer's disease.

The inventors of the present application have discovered that Posaconazole, when formulated as delayed-release tablets, offers a novel therapeutic approach for the treatment of Alzheimer's disease by modulating neuroinflammation, protecting neurons from oxidative stress, and preventing or slowing the progression of cognitive decline.

### SUMMARY OF THE INVENTION

The present invention provides a new therapeutic use of Posaconazole in the treatment of Alzheimer's disease. Specifically, Posaconazole, when administered as a delayed-release tablet, may exert beneficial effects by:
1. Reducing Neuroinflammation: Posaconazole may attenuate the inflammatory processes in the brain associated with Alzheimer's disease, inhibiting the activation of microglia and astrocytes, thereby reducing neuroinflammation.
2. Protecting Neurons from Oxidative Stress: Posaconazole has been found to possess antioxidant properties, which can protect neurons from oxidative stress, a key mechanism involved in Alzheimer's disease pathology.
3. Modulating Amyloid-Beta Accumulation: Posaconazole may help reduce the accumulation of amyloid-beta plaques, which are thought to play a central role in the pathogenesis of Alzheimer's disease.
4. Improving Cognitive Function: By reducing inflammation, oxidative stress, and amyloid-beta accumulation, Posaconazole may help improve cognitive function and memory in patients with Alzheimer's disease.

In one embodiment, the delayed-release formulation of Posaconazole ensures that the active compound is released gradually over an extended period, thereby providing sustained therapeutic effects with reduced systemic side effects compared to traditional immediate-release formulations.

1. A method of treating Alzheimer's disease in a patient, comprising administering a therapeutically effective amount of Posaconazole in the form of a delayed-release tablet.

2. The method of claim 1, wherein the delayed-release tablet releases Posaconazole gradually over a period of time.

3. The method of claim 1, wherein the patient is administered a daily dose of Posaconazole ranging from 100 mg to 600 mg.

4. The method of claim 1, wherein Posaconazole reduces neuroinflammation, oxidative stress, and amyloid-beta accumulation in the brain of the patient.

5. The method of claim 1, wherein the administration of Posaconazole improves cognitive function and memory in the patient.
